(19) United States
(12) Patent Application Publication
(10) Pub. No.: US 2024/0312608 A1
(43) Pub. Date: Sep. 19, 2024

(54) Medical Service Support Method, Recording Medium, and Medical Service Support System

(71) Applicant: SHIMADZU CORPORATION, Kyoto (JP)

(72) Inventors: Haruo UNO, Kyoto-shi (JP); Ryuta HIROSE, Kyoto-shi (JP); Tetsuya YAGI, Nagoya-shi (JP); Shintaro OYAMA, Nagoya-shi (JP); Mitsutaka IGUCHI, Nagoya-shi (JP); Hiroshi MORIOKA, Nagoya-shi (JP); Keisuke OKA, Nagoya-shi (JP)

(73) Assignee: SHIMADZU CORPORATION, Kyoto (JP)

(21) Appl. No.: 18/606,220

(22) Filed: Mar. 15, 2024

(30) Foreign Application Priority Data
Mar. 17, 2023 (JP) 2023-042754

(51) Int. Cl. G16H 40/20

(52) U.S. Cl. CPC G16H 40/20

(57) ABSTRACT

A medical service support system obtains disease information on a disease from which a patient may be suffered, based on patient information provided by a primary doctor for the patient, and determines a reception of a matching request. In response to the matching request from the primary doctor, the medical service support system identifies at least one specialist from among a plurality of specialists, and provides the at least one identified specialist, with the obtained disease information and the patient information.

### CROSS REFERENCE TO RELATED APPLICATIONS

This nonprovisional application is based on Japanese Patent Application No. 2023-042754 filed with the Japan Patent Office on Mar. 17, 2023, the entire contents of which are hereby incorporated by reference.

### BACKGROUND OF THE INVENTION

### Field of the Invention

The present invention relates to support of medical service for infections and the like.

### Description of the Background Art

In contrast to doctors in large-scale hospitals, in medium-to small-scale hospitals and clinics, doctors to cover areas of specialization do not necessarily work, and a doctor at a clinic may have to provide, as a primary doctor, medical service in an area different from his/her own specialization. For support of such a doctor, Japanese Patent Laying-Open No. 2022-040689 discloses a system that selects a specialist who works in another hospital, establishes a chat room between a primary doctor and the specialist, and transmits text or images and moving images from the primary doctor for consultation with the specialist.

### SUMMARY OF THE INVENTION

There is a room, however, for improvement of the system disclosed in Japanese Patent Laying-Open No. 2022-040689. For example, the specialist has to prepare answer contents only based on a consultation request letter from a primary doctor or backgrounds of a patient. More specifically, when the specialist makes a determination about a disease affecting the patient, the specialist has to determine the disease to be included in the answer contents from among enormous candidates for the disease. Therefore, in order to obtain a more accurate answer, the primary doctor and the specialist have to communicate with each other many times and it takes time for the primary doctor to be ready for determining courses of medical service.

In addition, in Japanese Patent Laying-Open No. 2022-040689, the specialist is selected only based on a clinical division desired for consultation by the primary doctor and years of experience. Since the area of specialization of a doctor is subdivided in the same clinical division, a technique for selecting an appropriate doctor depending on a condition of a patient has been desired.

Specifically, conventionally, information cooperation between the primary doctor and the specialist has not sufficiently been promoted or an appropriate specialist has not been selected. Therefore, a technique for appropriately supporting cooperation between the primary doctor and the specialist has been desired.

A medical service support method according to one aspect of the present disclosure includes obtaining disease information on a disease from which a patient may be suffered, based on patient information provided by a primary doctor of the patient, determining a reception of a matching request from the primary doctor, identifying at least one specialist from among a plurality of specialists in response to the matching request from the primary doctor, and providing the at least one identified specialist, with the obtained disease information and the patient information.

A medical service support method according to another aspect of the present disclosure includes obtaining disease information on a disease that may be affecting a patient by inputting patient information provided by a primary doctor of the patient into an estimation model, determining whether a matching request has been obtained from the primary doctor, allowing a plurality of specialists to view the disease information when the matching request has been obtained from the primary doctor, and receiving applications for an advisor from the plurality of specialists.

A recording medium according to one aspect of the present disclosure has a medical service support program stored thereon in a non-transitory manner. The medical service support program causes at least one processor to perform the medical service support method described above by being executed by the at least one processor.

A medical service support system according to one aspect of the present disclosure includes at least one processor and a storage where a medical service support program is stored, and the medical service support program causes the at least one processor to perform the medical service support method described above by being executed by the at least one processor.

The foregoing and other objects, features, aspects and advantages of this invention will become more apparent from the following detailed description of this invention when taken in conjunction with the accompanying drawings.

### BRIEF DESCRIPTION OF THE DRAWINGS

FIG. 1 is a diagram for illustrating provision of information by using a medical service support system 100.

FIG. 2 is a diagram showing exemplary generated structured data.

FIG. 3 is a diagram showing a hardware configuration of medical service support system 100.

FIG. 4 is a diagram showing an exemplary data structure in a specialist information database 240.

FIG. 5 is a diagram showing a mathematical expression showing an exemplary estimation model.

FIG. 6 is a flowchart of a process for matching of a primary doctor with at least one specialist.

FIG. 7 is a diagram showing an exemplary initial screen.

FIG. 8 is a diagram showing an exemplary screen that shows electronic medical chart data inputted by the primary doctor.

FIG. 9 is a diagram showing an exemplary screen updated as instructed in step S60.

FIG. 10 is a flowchart of a process performed by medical service support system 100.

### DESCRIPTION OF THE PREFERRED EMBODIMENTS

An embodiment of the present disclosure will be described in detail below with reference to the drawings. The same or corresponding elements in the drawings have the same reference characters allotted and description thereof will not be repeated.

### First Embodiment

### [Overall Configuration]

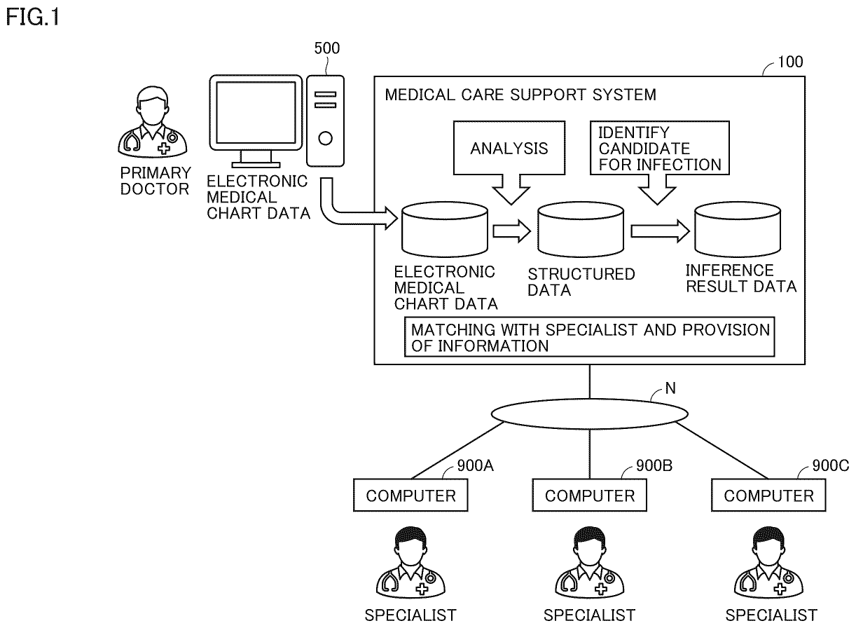

FIG. 1 is a diagram for illustrating provision of information by using a medical service support system 100. In a situation as shown in FIG. 1, a primary doctor is an infection non-specialist who sees a patient unclear as to whether or not he/she is affected by an infection, and the primary doctor operates an information terminal 500.

Information terminal 500 can communicate with medical service support system 100. When the primary doctor sees a patient, in order to seek for advice from a specialist, the primary doctor operates information terminal 500 to output a matching request to medical service support system 100.

When medical service support system 100 obtains the matching request from information terminal 500, it matches at least one specialist among a plurality of specialists with the primary doctor. Medical service support system 100 obtains patient information (electronic medical chart data or the like) from information terminal 500 and provides the patient information to the matched specialist.

More specifically, medical service support system 100 communicates with computers 900A, 900B, and 900C used by respective specialists over a network N. When computers 900A, 900B, and 900C are mentioned in connection with a common feature, that is, use by the specialist, they may be referred to as a “computer 900.” In one implementation, medical service support system 100 transmits the patient information to computer 900 associated with the matched specialist. The “computer associated with the matched specialist” can be identified, for example, as a computer in which the matched specialist has logged by an account name associated therewith. Though three computers (specialists) communicate over network N in an example in FIG. 1, the number of specialists in the medical service support system according to the present disclosure is not limited thereto.

Each of the at least one specialist who has obtained the patient information provides the primary doctor with advice for medical service for the patient. Advice may be provided with medical service support system 100 being interposed. Advice may be provided without medical service support system 100 being interposed. More specifically, the specialist may provide the primary doctor with advice by directly meeting the primary doctor, writing a letter to the primary doctor, and/or using communication means (telephone, electronic mail, in-house chat, or the like).

Medical service support system 100 may obtain the electronic medical chart data from information terminal 500 and generate candidates for a disease (infection) that may be affecting the patient. Medical service support system 100 may then use the candidates for the infection in identification of a specialist to be matched with the primary doctor. In generation of candidates for the infection, medical service support system 100 generates structured data by analyzing the electronic medical chart data. The structured data includes at least one item relating to a patient and a value of the at least one item.

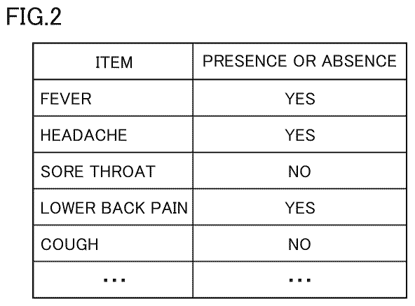

FIG. 2 is a diagram showing exemplary generated structured data. In the example shown in FIG. 2, the structured data includes at least five items. The structured data includes values of five respective items shown in FIG. 2. An exemplary item is “fever” and an exemplary value is “YES” for the item “fever”. In one implementation, the value of the item “fever” in the structured data for one patient is “YES” when the patient's body temperature is equal to or higher than 37.0° C., and the value is “NO” when the body temperature is lower than 37.0° C.

### [Hardware Configuration]

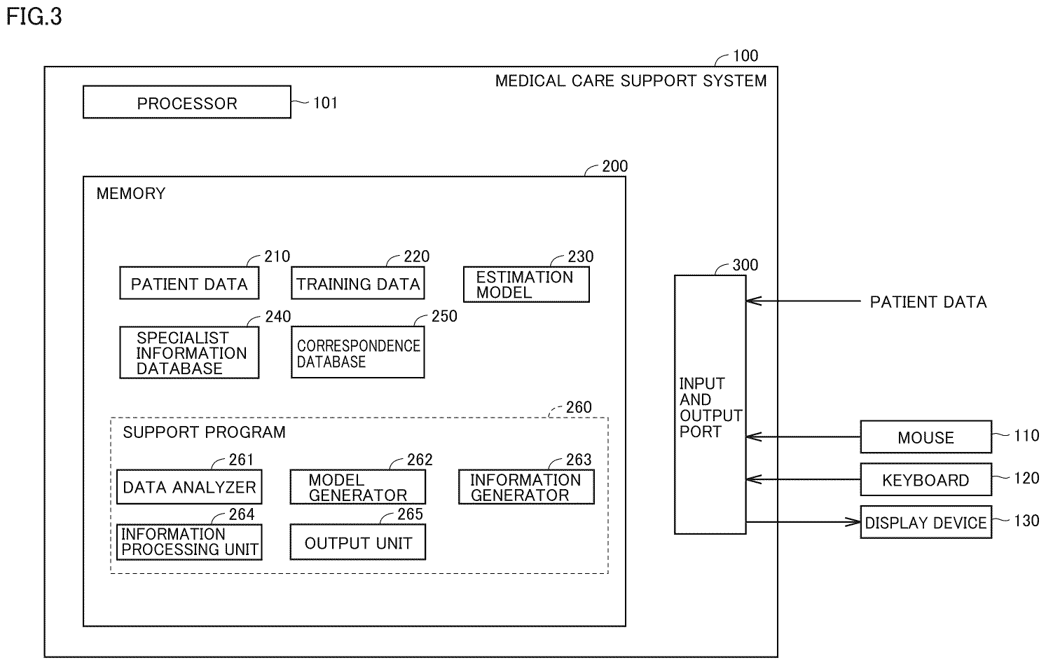

FIG. 3 is a diagram showing a hardware configuration of medical service support system 100.

Medical service support system 100 is configured, for example, based on a personal computer. Medical service support system 100 may be configured with a server accessible from at least one information terminal over a network such as the Internet.

Medical service support system 100 includes a processor 101, a memory 200, and an input and output port 300. A mouse 110, a keyboard 120, and a display device 130 are connected to input and output port 300.

Input and output port 300 may be a communication interface for data communication over the network. Patient data is inputted to input and output port 300. The patient data is, for example, electronic medical chart data. In one implementation, the structured data is generated from the electronic medical chart data. The structured data may be inputted to input and output port 300. Specifically, the primary doctor may extract a value of the at least one item included in the structured data from the electronic medical chart data and input the value of the at least one item to input and output port 300 through information terminal 500.

Patient data 210, training data 220, an estimation model 230, a specialist information database 240, a correspondence database 250, and a support program 260 are stored in memory 200 in a non-transitory manner.

Training data 220 is data to be used for machine learning by estimation model 230, training data 220 relating to a plurality of persons. In training data 220, the structured data as described with reference to FIG. 2 for each of the plurality of persons is tagged with infection affection information of each person. The affection information includes presence or absence of affection by an infection and a type of the affecting infection.

Estimation model 230 is a program for performing calculation in accordance with a model. Though an exemplary algorithm of estimation model 230 is logistic regression, the algorithm applied in estimation model 230 is not limited thereto. In one implementation, estimation model 230 is prepared for each of at least two types of infections, and the estimation model for each infection derives whether or not a patient is affected by the infection. The estimation model for each infection may derive a probability (certainty).

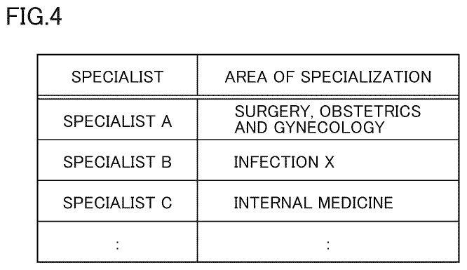

Information that identifies a clinical division in which each specialist specializes is stored in specialist information database 240. FIG. 4 is a diagram showing an exemplary data structure in specialist information database 240. As shown in FIG. 4, in specialist information database 240, an area of specialization (“surgery”, “obstetrics and gynecology,” “infection X,” “internal medicine,” . . . ) is stored. The area of specialization may be information that identifies the clinical division (for example, “surgery”) or the type of the infection (for example, “infection X”).

A name of an infection belonging to each of the at least two clinical divisions is stored in correspondence database 250.

Support program 260 includes, as its function, a data analyzer 261, a model generator 262, an information generator 263, an information processing unit 264, and an output unit 265. In one implementation, each of functions of data analyzer 261, model generator 262, information generator 263, information processing unit 264, and output unit 265 is performed by execution of a given program by processor 101.

Data analyzer 261 creates the structured data as described with reference to FIG. 2 from patient data 210.

In one implementation, data analyzer 261 extracts a portion in a pre-registered pattern from text of electronic medical chart data stored as patient data 210 by applying analysis processing such as morphemic analysis to the text. The “pre-registered pattern” refers to a pattern corresponding to a value of the structured data. Data analyzer 261 then recognizes the meaning of the portion in the extracted pattern in the electronic medical chart data and identifies a value corresponding to the recognized meaning. Data analyzer 261 then generates the structured data by combining the at least one identified value.

An exemplary registered pattern is “the body temperature is xx° C.” and a portion “xx” means a numerical value. When the electronic medical chart data includes text “the body temperature is 37.5° C.,” data analyzer 261 extracts the text “the body temperature is 37.5° C.” as the portion in the pre-registered pattern. Data analyzer 261 then recognizes that the body temperature of the patient is 37.5° C. as the meaning of the text. Data analyzer 261, on the other hand, is set such that the item and the value thereof in the structured data are identified based on the recognized meaning. For example, setting is made such that when the body temperature is equal to or higher than 37.0° C., the item “fever” and the value thereof “YES” are identified, and when the body temperature is lower than 37.0° C., the item “fever” and the value thereof “NO” are identified. Thus, when the electronic medical chart data includes the text “the body temperature is 37.5° C.,” data analyzer 261 generates the structured data including the value “YES” of the item “fever”.

The item of the structured data includes at least one of patient interview information, physical finding, and an examination result. The patient interview information, the physical finding, and the examination result may be included in the electronic medical chart data.

The patient interview information includes information obtained from a patient in an interview with the patient by the primary doctor. The patient interview information may include information obtained from the patient in an interview with the patient by another doctor in the past. Exemplary items included in the patient interview information include a main complaint of the patient.

The physical finding includes information collected from the patient by the primary doctor in medical examination (visual examination, auscultation, palpation, percussion, tendon reflex, light reflex, or the like).

The examination result includes a result of an examination conducted on the patient (a blood test, computed tomography (CT) scan, body temperature measurement, weight measurement, blood pressure measurement, heart rate measurement, or the like).

The item of the structured data may further include basic information of the patient (age, sex, a degree of necessity of care, a history of smoking, or the like), allergy information, and/or an item relating to anamnesis.

Model generator 262 uses training data 220 to perform machine learning processing on estimation model 230. Estimation model 230 subjected to machine learning processing is herein also referred to as a “trained model.”

Information generator 263 derives a result of determination as to an infection that may be affecting the patient by applying the structured data generated from the patient data to the trained model. In one implementation, information generator 263 uses the trained model for each type of the infection to derive the probability (certainty) that the patient is affected by each infection, and identifies as a candidate for the infection, the infection the probability of which is equal to or larger than a given value.

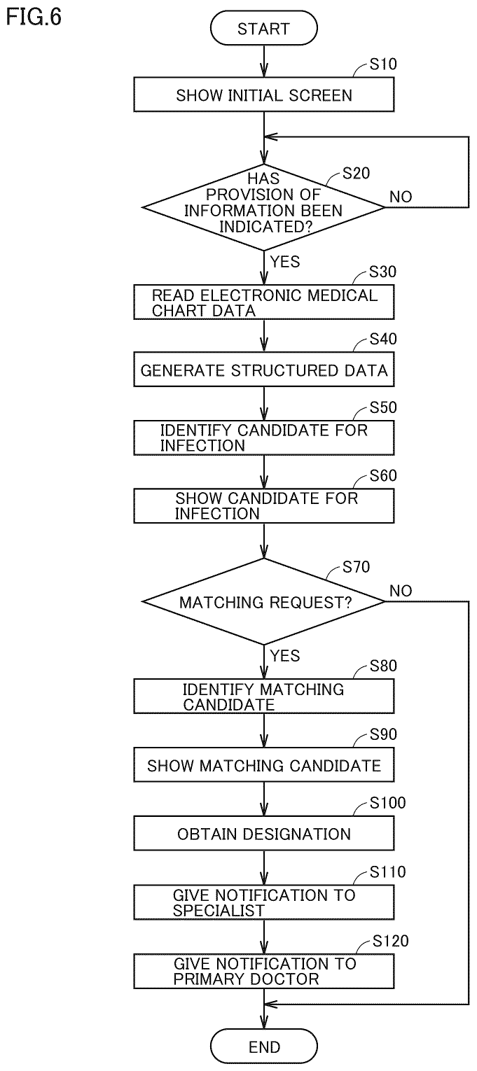

Information processing unit 264 performs processing for matching the primary doctor with at least one specialist. Contents of the processing for matching will be described later with reference to FIG. 6 or the like.

Output unit 265 generates image information for showing information identified in support program 260 and instructs information terminal 500 to show the image information.

### [Identification of Candidate for Infection]

One implementation of identification of a candidate for an infection by information generator 263 will be described. As described above with reference to FIG. 3, in one implementation, estimation model 230 is prepared for each type of the infection and information generator 263 derives the probability that the patient is affected by each infection by applying the structured data to each estimation model 230.

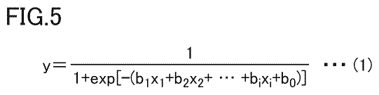

FIG. 5 is a diagram showing a mathematical expression showing an exemplary estimation model. An expression (1) shown in FIG. 5 complies with logistic regression analysis. In the expression (1), a subscript represents the type of the item included in the structured data. In the expression (1), i types of items from “1” to “i” are defined. A value of “i” may be different depending on the type of the infection. Specifically, the type and the number of items to be used for estimation of the probability of affection by each infection may be different for each infection. Information that associates each infection with i types of items included in the expression (1) prepared for each infection is stored in correspondence database 250.

In the expression (1), x represents an explanatory variable and corresponds to the value of each item in the structured data. For example, when the value is “YES” for the item “fever”, x is 1, and when the value is “NO”, x is 0.

In the expression (1), b represents a coefficient. A value of each of b₀ to bᵢ is set in machine learning for each infection.

In the expression (1), y represents an objective variable and represents a result of calculation for each infection. The value of y represents certainty of affection by each infection.

Information generator 263 derives certainty that the patient is affected by each infection by application of the value of each item included in the structured data to the estimation model for each infection.

Information generator 263 then identifies the infection the value of “certainty” of which is equal to or larger than a given threshold value, as a candidate for the infection that is affecting the patient.

A manner of identification of the candidate for the infection described with reference to FIG. 4 is merely by way of example. The candidate for the infection may be identified in any other type of manner such as multivariable analysis where at least one type included in the structured data is defined as the variable.

In application of the structured data to the estimation model, in connection with at least one infection, the value of at least one of items defined in the estimation model may not be included in the structured data. In other words, at least one of the items defined in the estimation model may be missing in the structured data.

An example in which the estimation model for an infection “pyelonephritis” defines three types of items (fever, frequent urination, and flank pain) is assumed. When the primary doctor incorporates information on fever and frequent urination but does not incorporate information on flank pain in electronic medical chart data, the structured data includes values of the item “fever” and the item “frequent urination” but does not include the value of the item “flank pain.” In such a case, information generator 263 performs missing value processing in derivation of certainty with the use of the estimation model. Exemplary missing value processing is random complement of the value of the missing item with a value of another item to be used in the expression of the same estimation model. Another example is complement of the value of the missing item with an average value of values of other items to be used in the expression of the same estimation model. For example, when the value of the item “flank pain” is missing, this value is complemented by the average value of the value of the item “fever” and the value of the item “frequent urination.”

### [Important Factor]

In memory 200, a score of importance of each item calculated at the time of generation of the estimation model may be stored for each infection. For example, an item having the score of the importance equal to or larger than a given value among items included in the structured data may be identified as the important factor for each infection.

“Being equal to or larger than a given value” may mean that the score of the importance has a “positive” value equal to or larger than a certain value.

“Being equal to or larger than a given value” may mean that an absolute value of the score of the importance is equal to or larger than a given value. When the score of the importance of a certain item has a positive value, magnitude of the absolute value of the score represents likelihood of infection. When the score of the importance of a certain item has a negative value, magnitude of the absolute value of the score represents unlikelihood of infection. When “being equal to or larger than a given value” means that the absolute value of the score of the importance is equal to or larger than a given value, the item identified as the important factor characterizes likelihood of infection or unlikelihood of infection.

### [Flow of Process]

FIG. 6 is a flowchart of a process for matching of a primary doctor with at least one specialist. In one implementation, the process in FIG. 6 is performed by execution of a given program (medical service support program) by processor 101 of medical service support system 100.

In one implementation, the process in FIG. 6 is started in response to a request from information terminal 500 to medical service support system 100 to start provision of information. In one implementation, information terminal 500 requests medical service support system 100 to start provision of information in response to start-up of an application program for medical service support and an operation to request start of provision of information in the application program.

Referring to FIG. 6, in step S10, medical service support system 100 instructs information terminal 500 to show an initial screen. Information terminal 500 thus shows the initial screen on the display thereof.

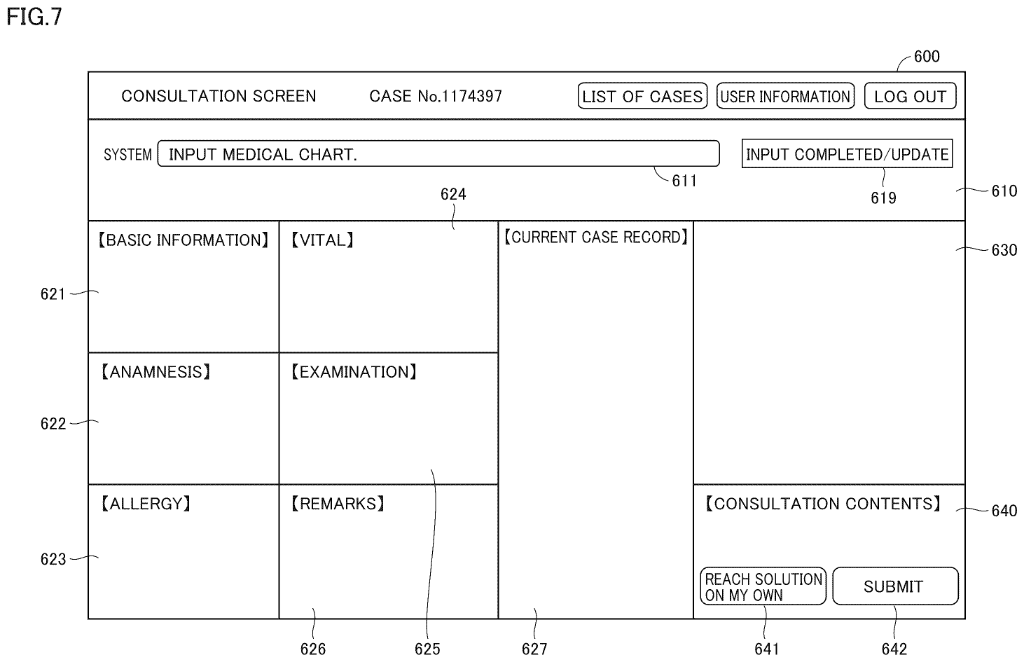

FIG. 7 is a diagram showing an exemplary initial screen. In FIG. 7, a screen 600 includes a first communication frame 610, fields 621 to 627 and 630, and a second communication frame 640.

First communication frame 610 shows information for communication by the primary doctor with medical service support system 100. First communication frame 610 includes a field 611 and a button 619. In the example in FIG. 6, a message (input medical chart) to the primary doctor is shown in field 611. Button 619 is operated to instruct medical service support system 100 to provide information for data inputted in the screen.

Electronic medical chart data of a patient is inputted in each of fields 621 to 627. More specifically, basic information (age, sex, a degree of necessity of care, a history of smoking, or the like) of the patient is inputted in field 621. Anamnesis of the patient is inputted in field 622. Allergy information of the patient is inputted in field 623. Vital information among results of examinations conducted on the patient is inputted in field 624. A result of an examination (a result of a blood test, a result of CT scan, or the like) other than the vital information among the results of examinations conducted on the patient is inputted in field 625. Physical finding is inputted in field 626. Patient interview information (a main complaint or the like) is inputted in field 627. A manner of input of the electronic medical chart data in the description above is merely by way of example. Medical service support system 100 may receive input of the electronic medical chart data described in a text format and may generate screen data as shown in FIG. 6 by extracting data to be shown in fields 621 to 627 from the electronic medical chart data.

In field 630, information on candidates for an infection that may be affecting the patient is shown, the information being provided from medical service support system 100 to the primary doctor.

Second communication frame 640 is used for the primary doctor to interact with an infection specialist.

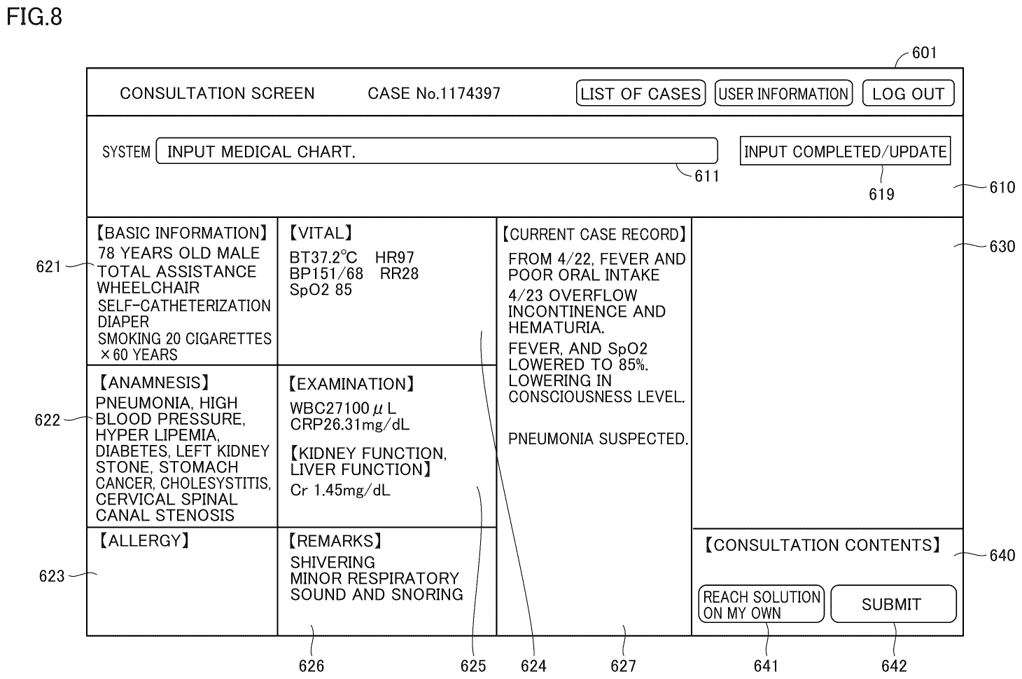

The primary doctor who visually recognizes screen 600 inputs the electronic medical chart data in fields 621 to 627. FIG. 8 is a diagram showing an exemplary screen that shows the electronic medical chart data inputted by the primary doctor. In FIG. 8, a screen 601 shows the electronic medical chart data inputted by the primary doctor in each of fields 621 to 627.

Thereafter, the primary doctor operates button 619. The inputted electronic medical chart data is thus transmitted from information terminal 500 to medical service support system 100.

Referring to back to FIG. 6, in step S20, medical service support system 100 determines whether or not it has been instructed to provide information by information terminal 500. Medical service support system 100 repeats control in step S20 until it determines that it has been instructed to provide information (NO in step S20). When medical service support system 100 determines that it has been instructed to provide information (YES in step S20), it allows control to proceed to step S30.

In step S30, medical service support system 100 reads the electronic medical chart data transmitted from information terminal 500.

In step S40, medical service support system 100 generates the structured data as described with reference to FIG. 2 by using the electronic medical chart data read in step S30.

In step S50, medical service support system 100 identifies at least one candidate for an infection that may be affecting the patient by using the structured data generated in step S40.

In step S60, medical service support system 100 generates a display screen to show information including candidates for the infection identified in step S50 and instructs information terminal 500 to show the generated display screen. The display screen is thus updated in information terminal 500.

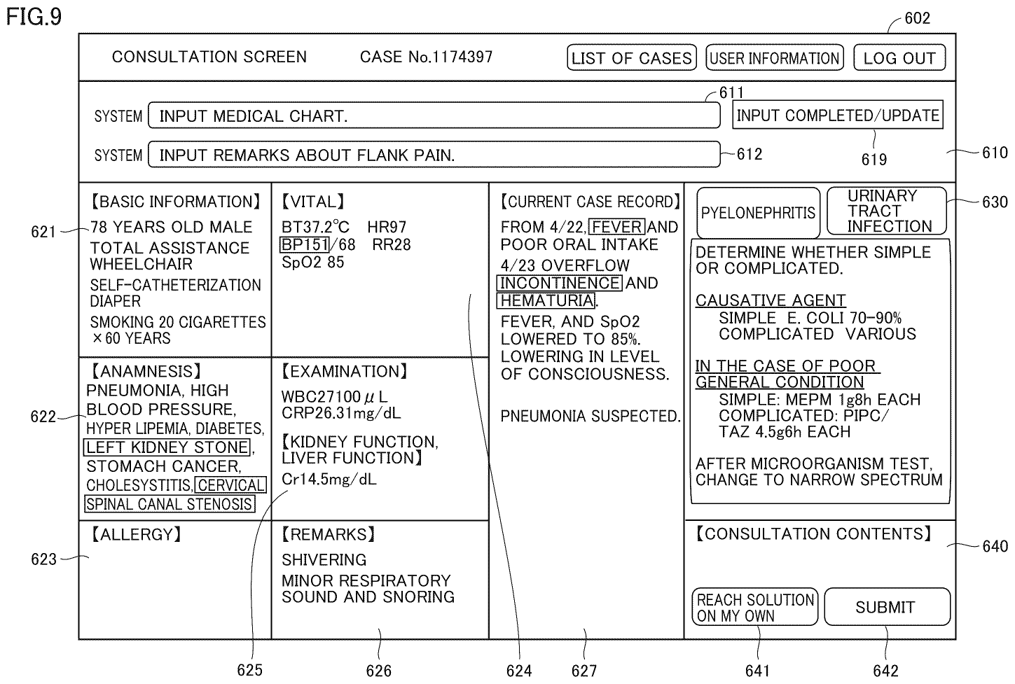

FIG. 9 is a diagram showing an exemplary screen updated as instructed in step S60. In FIG. 9, a screen 602 is an exemplary screen after update of screen 601 in FIG. 8.

As compared with screen 601 in FIG. 8, in screen 602 in FIG. 9, at least one candidate for the infection that may be affecting the patient is shown in field 630. At least one tab shown in field 630 corresponds to at least one respective candidate for the infection identified in step S50.

In field 630 in FIG. 9, two tabs showing two respective types of infections (pyelonephritis and urinary tract infection) are shown.

More specifically, in field 630, two tabs are shown to switch contents to be shown. FIG. 9 shows a state in which, of the tab of “pyelonephritis” and the tab of “urinary tract infection,” contents in the tab “pyelonephritis” are shown. The primary doctor can have contents in the tab of “urinary tract infection” shown in field 630 by operating information terminal 500.

Contents in each tab in field 630 are medical service support information of a corresponding infection. In step S50, medical service support system 100 may obtain medical service support information of each obtained candidate for the infection and have the medical service support information shown in each tab in field 630.

As compared with FIG. 8, in FIG. 9, a field 612 is added to first communication frame 610. In step S50, medical service support system 100 may identify for each candidate for the infection, an item that is lacking (lacking item) for improvement in certainty of the candidate, or may add a field to first communication frame 610 and have a message shown in the added field, the message inviting input of a value of the lacking item.

In the example in FIG. 9, as compared with FIG. 8, a part of text shown in fields 621 to 627 is shown as being emphasized by being enclosed by a frame. The text shown as being emphasized corresponds to the important factor. In step S50, medical service support system 100 may identify the important factor for each candidate for the infection or may have a portion corresponding to the important factor in the text (electronic medical chart data) shown in fields 621 to 627 shown as being emphasized. The portion corresponding to the important factor is a portion of the electronic medical chart data used for generation of the value of the item identified as the important factor in generation of the structured data.

An example in which the infection “pyelonephritis” is identified as the candidate, correspondence database 250 defines three types of items (fever, frequent urination, and flank pain) as the items to be used for estimation of the infection “pyelonephritis,” and “fever” and “frequent urination” are identified as the important factors is assumed. In this case, the structured data further includes the value “YES” of the item “fever” generated from text “BT 37.2° C.” in the electronic medical chart data and the value “YES” of the item “frequent urination” generated from text “increase in number of times of discharge of urine” in the electronic medical chart data. In this case, a portion corresponding to the important factor “fever” in the electronic medical chart data is “BT 37.2° C.” and a portion corresponding to the important factor “frequent urination” is “increase in number of times of discharge of urine.” Therefore, in the case above, in fields 621 to 627, “BT 37.2° C.” and “increase in number of times of discharge of urine” in the electronic medical chart data are shown as being emphasized.

Referring back to FIG. 6, in step S70, medical service support system 100 determines whether or not it has obtained the matching request from information terminal 500.

The matching request refers to information on a request for matching with a specialist from the primary doctor. In one implementation, the primary doctor operates a button 642 in second communication frame 640 on the screen (screen 602 or the like) to transmit the matching request from information terminal 500 to medical service support system 100.

When medical service support system 100 determines that it has obtained the matching request (YES in step S70), it allows control to proceed to step S80. When medical service support system 100 determines that it has not obtained the matching request (NO in step S70), it quits the process in FIG. 6. In one implementation, when medical service support system 100 instructs information terminal 500 to show a screen in step S60 and thereafter button 642 is not operated for a certain time period or when a button 641 is operated, it may determine that it has not obtained the matching request.

In step S80, medical service support system 100 identifies a matching candidate. More specifically, medical service support system 100 refers to specialist information database 240 and identifies as the matching candidate, a specialist the area of specialization of which includes the candidate for the infection identified in step S50. An example of including the candidate for the infection as the area of specialization is that the type itself of the infection identified as the candidate is included in the area of specialization. Another example is that the clinical division corresponding to the type of the infection identified as the candidate is included in the area of specialization. Information that defines correspondence between the type of the infection and the clinical division may be stored in memory 200. Medical service support system 100 may identify the clinical division corresponding to the type of the infection identified as the candidate, by referring to this information.

In step S90, medical service support system 100 generates a screen where the matching candidate identified in step S80 is to be shown and instructs information terminal 500 to show the screen. The primary doctor who uses information terminal 500 thus recognizes the matching candidate.

The primary doctor operates information terminal 500 to designate the specialist as his/her advisor from at least one specialist shown as the matching candidate. In response, information terminal 500 transmits information designating the specialist to medical service support system 100.

In step S100, medical service support system 100 obtains the information that designates the specialist from information terminal 500.

In step S110, medical service support system 100 notifies the designated specialist of designation as the advisor of the primary doctor. At this time, medical service support system 100 transmits the patient information (electronic medical chart data or the like: step S30) and the candidate for the infection that may be affecting the patient (step S50) to computer 900 of the designated specialist.

In step S120, medical service support system 100 notifies information terminal 500 of information on the designated specialist. In response, information terminal 500 shows the information on the designated specialist. The primary doctor can thus recognize the information (for example, a contact or the like which will be described later) on the designated specialist. Thereafter, medical service support system 100 quits the process in FIG. 6.

As set forth above, in the process described with reference to FIG. 6, the candidate for the specialist is identified based on the candidate for the infection that may be affecting the patient, the candidate being estimated from the patient information, and information on each specialist registered in specialist information database 240. Then, the specialist designated by the primary doctor is finally identified as the advisor of the primary doctor from among the candidates for the specialist. Thus, in the present embodiment, the primary doctor can obtain the advisor by being matched with at least one specialist.

Designation by the primary doctor does not have to be made. Specifically, in the process in FIG. 6, steps S90 to S110 may be skipped and the candidate for the specialist identified in step S80 may automatically be identified as a final advisor.

Use of the estimation model for identification of the candidate for the infection that may be affecting the patient is not essential. The candidate may be identified on a rule basis. Specifically, for example, information that associates a keyword and the infection with each other may be stored in memory 200. Medical service support system 100 may then identify the infection associated with the keyword included in the electronic medical chart data obtained in step S30 (or the structured data generated in step S40) as the candidate for the infection that may be affecting the patient, by referring to the information.

The information given in step S120 may include contact information (a telephone number, a mail address, or the like) of the specialist designated as the advisor. The primary doctor can thus contact the specialist designated as the advisor without medical service support system 100 being interposed.

The primary doctor, on the other hand, can contact the specialist designated as the advisor with medical service support system 100 being interposed. For example, after step S120, medical service support system 100 can transmit information (question contents or the like) transmitted from information terminal 500 of the primary doctor to computer 900 of the specialist or transmit an answer transmitted from computer 900 of the specialist to information terminal 500 of the primary doctor.

The primary doctor may input consultation contents in second communication frame 640 before operation onto button 642. Thus, as a result of operation onto button 642, the consultation contents inputted in second communication frame 640 are transmitted to medical service support system 100 together with the matching request. Medical service support system 100 may transfer the consultation contents transmitted from information terminal 500 to computer 900.

The specialist may introduce another specialist to the primary doctor as a result of contact with the primary doctor. For example, the answer transmitted from computer 900 of the specialist may include information that identifies another specialist. The primary doctor can thus consult another specialist for diagnosis of the patient. In this case, medical service support system 100 may transmit the electronic medical chart data obtained from information terminal 500 to computer 900 of another specialist. Medical service support system 100 may further transmit communication contents (consultation contents) between the primary doctor and the specialist to computer 900 of another specialist.

In identification of the matching candidate, in addition to or instead of the area of specialization of the specialist, at least another type of information (or combination of at least two types of information) on the specialist may be used. When the area of specialization of the specialist is not used, identification of the candidate for the infection in step S50 may be skipped.

Another exemplary type of information on the specialist is information that identifies date and time when each specialist can attend. In this case, in step S80, medical service support system 100 may identify as the candidate for the advisor, a specialist who satisfies a condition that date and time when the specialist can attend includes timing of determination as obtainment of matching information in step S70. Another exemplary type of information on the specialist is information that identifies a time period required by each specialist to respond. In this case, the primary doctor may input information designating the time period before obtainment of the response in information terminal 500 at the time of operation onto button 642. Information terminal 500 transfers such information to medical service support system 100. In step S80, medical service support system 100 may identify as the candidate for the advisor, a specialist who requires a time period to respond equal to or shorter than the time period before obtainment of the response designated by the primary doctor.

Another exemplary type of information on the specialist is information that identifies the number of primary doctors to which each specialist can attend in a given period. In this case, a history of being identified as the advisor may be stored in memory 200 for each specialist. In step S80, medical service support system 100 may identify as the candidate for the advisor, a specialist of which number of times of being identified as the advisor in a given immediately preceding period has not reached the number above. For example, when “five” is registered as the number of primary doctors (an upper limit of the number of times that the specialist can serve as the advisor) to which a specialist A can attend in one month, medical service support system 100 identifies specialist A as the candidate for the advisor on condition that the number of times of identification of specialist A as the advisor in immediately preceding one month is four or smaller.

Another exemplary type of information on the specialist is information on each specialist that identifies years of experience as the specialist. In this case, the primary doctor may input the minimum years of experience in information terminal 500 as a condition for the advisor in operation onto button 642. Information terminal 500 transfers the minimum years of experience to medical service support system 100. In step S80, medical service support system 100 may identify a specialist having the years of experience equal to or longer than the minimum years of experience as the candidate for the advisor.

Another exemplary type of information on the specialist may be information that identifies the number of primary doctors to which each specialist is attending. In step S80, at the timing of determination as obtainment of the matching request in step S70, medical service support system 100 may identify as the candidate for the advisor, a specialist who is attending to primary doctor(s) in number smaller than a given threshold value at that time point.

In one implementation, information for management of activities by each specialist, that is, for which primary doctor each specialist is serving as the advisor, is registered in specialist information database 240. For example, when specialist A is identified as the advisor of a primary doctor X (or specialist A is designated as the advisor by primary doctor X), medical service support system 100 registers information on the specialist who indicates “being attending to: primary doctor X” in specialist information database 240. When specialist A finishes attendance to primary doctor X, specialist A uses computer 900 to transmit information indicating end of attendance on primary doctor X to medical service support system 100. Medical service support system 100 can recognize to how many primary doctors each specialist is attending, by referring to specialist information database 240.

Another exemplary type of information on the specialist is information that identifies evaluation of each specialist. In this case, the primary doctor may input minimum required evaluation (for example, points) in information terminal 500 as a condition for the advisor in operation onto button 642. Information terminal 500 transfers the minimum evaluation to medical service support system 100. In step S80, medical service support system 100 may identify a specialist who has evaluation equal to or higher than the minimum evaluation as the candidate for the advisor. Evaluation of each specialist may vary. Medical service support system 100 may identify the candidate for the advisor in accordance with evaluation of each specialist at the time point of determination as obtainment of the matching request.

Another exemplary type of information on the specialist may be position information of each specialist. Each specialist registers in specialist information database 240, for example, a location of a hospital where the specialist mainly works as the position information. In this case, medical service support system 100 may obtain position information of the primary doctor (for example, a location of a hospital where the primary doctor sees the patient at that time point) together with the matching request from information terminal 500. Then, in step S80, medical service support system 100 may identify as the candidate for the advisor, the specialist who has the position information that satisfies a specific condition for the position of the primary doctor. The specific condition is, for example, “being closest to the primary doctor.” In this case, when three specialists A, B, and C are primarily identified as the candidates based on the candidates for the infection that may be affecting the patient, medical service support system 100 may identify as the candidate for the advisor, the specialist who has information that identifies the position closest to the position of the primary doctor as the position information, among specialists A, B, and C.

In the present embodiment, the primary doctor can be referred to a specialist simply by transmission of electronic medical chart data to medical service support system 100. Burdens imposed on the primary doctor for search for a specialist can thus drastically be lessened. In particular, such great burdens as observation of the patient have already been imposed on the primary doctor. Therefore, the technique in the present disclosure capable of thus lessening burdens imposed on the primary doctor is greatly significant.

In the present embodiment, the electronic medical chart data is analyzed in natural language processing to generate the structured data. The primary doctor may generate the structured data based on contents of the electronic medical chart data. As the structured data is generated in natural language processing of the electronic medical chart data, appropriate structured data can reliably be generated even when the primary doctor does not understand a method of generating the structured data for diagnosis of the infection.

In the first embodiment described above, when the primary doctor of the patient who is the infection non-specialist outputs the matching request, at least one specialist (advisor) is identified and the candidate for the infection that may be affecting the patient is provided to at least one identified specialist. Information necessary for the advisor is thus provided to the at least one identified specialist. Therefore, cooperation between the primary doctor and the specialist is appropriately supported.

### Second Embodiment

In the first embodiment, as described with reference to FIG. 6, medical service support system 100 receives from the primary doctor, designation of the final advisor from candidates for the advisor. In a second embodiment, medical service support system 100 receives applications for the final advisor from specialists identified as candidates for the advisor.

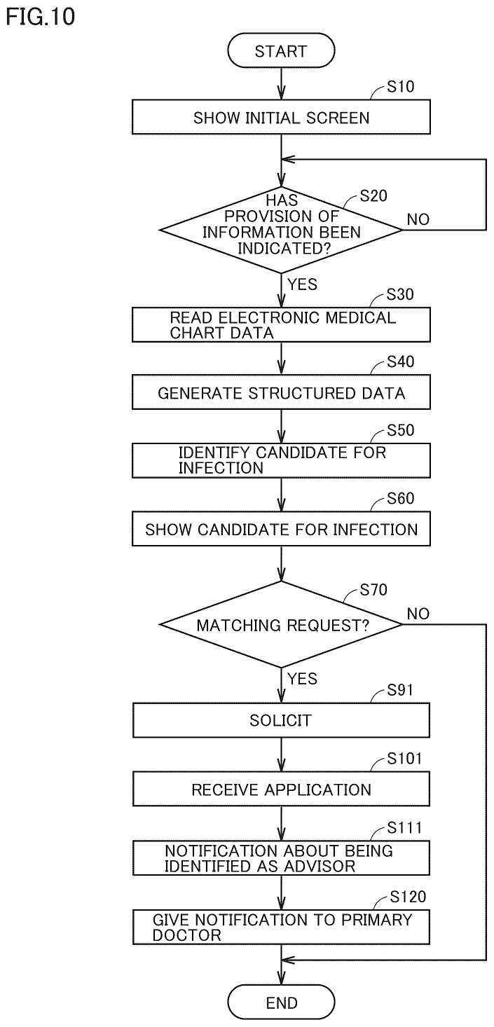

FIG. 10 is a flowchart of a process performed by medical service support system 100. As compared with the process in FIG. 6, the process in FIG. 10 includes steps S91, S101, and S111 instead of steps S80, S90, S100, and S110.

More specifically, in the process in FIG. 10, when medical service support system 100 determines that it has obtained the matching request in step S70 (YES in step S70), it carries out control in step S91. In step S91, medical service support system 100 notifies computers 900 of all specialists of solicitation for the advisor of the primary doctor.

Medical service support system 100 transmits the notification in step S91 to computers 900 of all specialists, together with information on the primary doctor (a name of the primary doctor, a name of a hospital where the primary doctor works, or the like), electronic medical chart data, and/or at least one candidate for the infection that may be affecting the patient. When medical service support system 100 has obtained consultation contents (inputted in second communication frame 640) together with the matching request, it may transmit the notification in step S91 together with the consultation contents.

The specialist who sees the notification in step S91 determines whether or not he/she applies for the advisor and inputs a result in computer 900. Computer 900 transmits the result to medical service support system 100.

In step S101, medical service support system 100 receives application from the specialist by receiving the result transmitted from computer 900.

In step S111, medical service support system 100 identifies the advisor and notifies computer 900 of the specialist identified as the advisor of being identified as the advisor.

In step S120, medical service support system 100 notifies information terminal 500 of information about the specialist identified as the advisor. In response, information terminal 500 shows the information on the specialist identified as the advisor. Thereafter, medical service support system 100 quits the process in FIG. 10.

In the process described with reference to FIG. 10, the number of advisors to be identified in step S111 may be set in advance. Medical service support system 100 may close the application at the time point of reception of the set number of applications. In this case, all specialists that have applied are identified as the advisors. The number of advisors may be set by the primary doctor. For example, the primary doctor may input information designating the number of advisors in the screen (screen 602 or the like) when the primary doctor operates button 642. Information terminal 500 may transmit the inputted number to medical service support system 100 as the number of advisors.

In the second embodiment described above, when the primary doctor of the patient who is the infection non-specialist outputs the matching request, applications for the advisor are received from a plurality of specialists who have been notified of at least one candidate for the infection that may be affecting the patient. Each specialist can thus determine that the specialist himself/herself is the appropriate specialist as the advisor relating to the at least one candidate for the infection, and then apply for the advisor. Therefore, a specialist appropriate for the primary doctor is identified and cooperation between the primary doctor and the specialist is appropriately supported.

### <Modification>

Medical service support system 100 may identify the finally identified advisor based on both of designation from the primary doctor and application from the specialist.

For example, medical service support system 100 may primarily identify candidates for the advisor based on information registered in connection with specialists, thereafter notify specialists designated by the primary doctor from the candidates of being identified as the candidates, and thereafter receive application from the specialists who received the notification.

Medical service support system 100 may primarily identify candidates for the advisor based on information registered in connection with the specialist, thereafter transmit the notification to specialists identified as the candidates, receive applications from the specialists, present the specialists who have applied to the primary doctor, and receive from the primary doctor, information designating the final advisor from among presented specialists.

### <Aspects>

Illustrative embodiments described above are understood by a person skilled in the art as specific examples of aspects below.

(Clause 1) A medical service support method according to one aspect includes obtaining disease information on a disease from which a patient may be suffered, based on patient information provided by a primary doctor for the patient, determining a reception of a matching request from the primary doctor, identifying at least one specialist from among a plurality of specialists in response to the matching request has been obtained from the primary doctor, and providing the at least one identified specialist, with the obtained disease information and the patient information.

According to the medical service support method in Clause 1, cooperation between the primary doctor and the specialist is appropriately supported.

(Clause 2) In the medical service support method in Clause 1, the identifying at least one specialist may include identifying candidates for the specialist based on registration information of each of the plurality of specialists and the matching request, presenting the candidates to the primary doctor, and obtaining from the primary doctor, designation of the at least one specialist from the candidates.

According to the medical service support method in Clause 2, the primary doctor himself/herself can designate the specialist who is to cooperate with the primary doctor, and a specialist preferred for the primary doctor is designated.

(Clause 3) In the medical service support method in Clause 2, the registration information may include information that identifies an area of specialization, and the identifying at least one specialist may include identifying as the at least one specialist, a specialist who satisfies a condition that information corresponding to the disease information is registered as the area of specialization of the specialist.

According to the medical service support method in Clause 3, accuracy in matching between the primary doctor and the specialist can be improved, and a specialist appropriate for the disease information is thus identified as at least one specialist.

(Clause 4) In the medical service support method in Clause 3, the area of specialization may include at least one of a clinical division and a type of a disease.

According to the medical service support method in Clause 4, a specialist more appropriate for the disease information is identified as at least one specialist.

(Clause 5) In the medical service support method described in any one of Clauses 2 to 4, the registration information may include information that identifies date and time when each specialist can attend, and the identifying at least one specialist may include identifying as the at least one specialist, a specialist who satisfies a condition that date and time when the specialist can attend includes corresponding date and time in the matching request.

According to the medical service support method in Clause 5, the primary doctor can more reliably be supported by the identified specialist.

(Clause 6) In the medical service support method described in any one of Clauses 2 to 5, the registration information may include information that identifies a time period required by each specialist to respond, and the identifying at least one specialist may include identifying as the at least one specialist, a specialist who satisfies a condition that the information that identifies the time period required by the specialist to respond satisfies a request for the time period in the matching request.

According to the medical service support method in Clause 6, the primary doctor can more reliably be supported by the identified specialist.

(Clause 7) In the medical service support method described in any one of Clauses 2 to 6, the registration information may include information that identifies the number of primary doctors to which each specialist can attend in a given period, and the identifying at least one specialist may include identifying as the at least one specialist, a specialist who satisfies a condition that the number of primary doctors to which the specialist have attended in the given period is smaller than the number of primary doctors to which the specialist can attend, at the time when the matching request is obtained.

According to the medical service support method in Clause 7, the number of times that a specialist is identified as an advisor of the primary doctor can be balanced among a plurality of specialists.

(Clause 8) In the medical service support method described in any one of Clauses 2 to 7, the registration information may include information that identifies years of experience of each specialist, and the identifying at least one specialist may include identifying as the at least one specialist, a specialist who has the years of experience equal to or longer than the years of experience identified in the matching request.

According to the medical service support method in Clause 8, the primary doctor can be supported by the identified specialist in a manner desired by the primary doctor.

(Clause 9) In the medical service support method described in any one of Clauses 2 to 8, the registration information may include information that identifies the number of primary doctors to which each specialist is attending, and the identifying at least one specialist may include identifying the at least one specialist based on the number of primary doctors to which each specialist is attending at a time point when the matching request is obtained.

According to the medical service support method in Clause 9, the primary doctor can more reliably be supported by the identified specialist.

(Clause 10) In the medical service support method described in any one of Clauses 2 to 9, the registration information may include information that identifies evaluation of each specialist, and the identifying at least one specialist may include identifying the at least one specialist based on evaluation of each specialist.

According to the medical service support method in Clause 10, the primary doctor can be supported by the specialist who has characteristics desired by the primary doctor.

(Clause 11) In the medical service support method described in any one of Clauses 2 to 10, the registration information may include position information of each specialist, and the identifying at least one specialist may include identifying the at least one specialist based on position information of the primary doctor from which the matching request has been obtained and the position information of each specialist.

According to the medical service support method in Clause 11, the primary doctor can be supported by the identified specialist more reliably and/or in a manner desired by the primary doctor.

(Clause 12) A medical service support method according to one aspect includes obtaining disease information on a disease from which a patient may be suffered, based on patient information provided by a primary doctor for the patient, determining a reception of a matching request from the primary doctor, allowing a plurality of specialists to view the disease information in response to the matching request from the primary doctor, and receiving applications for an advisor from the plurality of specialists.

According to the medical service support method in Clause 12, cooperation between the primary doctor and the specialist is appropriately supported.

(Clause 13) In the medical service support method described in Clause 12, the allowing a plurality of specialists to view the disease information may include allowing the plurality of specialists to view the patient information.

According to the medical service support method in Clause 13, the specialist is provided with more detailed information for making determination as to whether or not to file an application.

(Clause 14) The medical service support method described in any one of Clauses 1 to 13 may further include providing the primary doctor with the disease information, and the determining the reception of the matching request may include determining whether the matching request has been received through an information terminal from the primary doctor provided with the disease information.

According to the medical service support method in Clause 14, the matching request is obtained only when the primary doctor provided with the disease information requires an advisor.

(Clause 15) A medical service support program according to one aspect may cause at least one processor to perform the medical service support method described in any one of Clauses 1 to 14 by being executed by the at least one processor.

According to the medical service support program in Clause 15, cooperation between the primary doctor and the specialist is appropriately supported.

(Clause 16) A medical service support system according to one aspect includes at least one processor and a storage where a medical service support program is stored, and the medical service support program may cause the at least one processor to perform the medical service support method described in any one of Clauses 1 to 14 by being executed by the at least one processor.

According to the medical service support system in Clause 16, cooperation between the primary doctor and the specialist is appropriately supported.

Though the embodiments of the present invention have been described, it should be understood that the embodiments disclosed herein are illustrative and non-restrictive in every respect. The scope of the present invention is defined by the terms of the claims and is intended to include any modifications within the scope and meaning equivalent to the terms of the claims.

What is claimed is:

1. A medical service support method comprising:
obtaining disease information on a disease from which a patient may be suffered, based on patient information provided by a primary doctor for the patient;
determining a reception of a matching request has been obtained from the primary doctor;
identifying at least one specialist from among a plurality of specialists in response to the matching request from the primary doctor; and
providing the at least one identified specialist, with the obtained disease information and the patient information.

2. The medical service support method according to claim 1, wherein
the identifying at least one specialist includes
identifying candidates for the specialist based on registration information of each of the plurality of specialists and the matching request,
presenting the candidates to the primary doctor, and
obtaining from the primary doctor, designation of the at least one specialist from the candidates.

3. The medical service support method according to claim 2, wherein
the registration information includes information that identifies an area of specialization, and
the identifying at least one specialist includes identifying as the at least one specialist, a specialist who satisfies a condition that information corresponding to the disease information is registered as the area of specialization of the specialist.

4. The medical service support method according to claim 3, wherein
the area of specialization includes at least one of a clinical division and a type of a disease.

5. The medical service support method according to claim 2, wherein
the registration information includes information that identifies date and time when each specialist can attend, and
the identifying at least one specialist includes identifying as the at least one specialist, a specialist who satisfies a condition that date and time when the specialist can attend includes corresponding date and time in the matching request.

6. The medical service support method according to claim 2, wherein
the registration information includes information that identifies a time period required by each specialist to respond, and
the identifying at least one specialist includes identifying as the at least one specialist, a specialist who satisfies a condition that the information that identifies the time period required by the specialist to respond satisfies a request for the time period in the matching request.

7. The medical service support method according to claim 2, wherein
the registration information includes information that identifies the number of primary doctors to which each specialist can attend in a given period, and
the identifying at least one specialist includes identifying as the at least one specialist, a specialist who satisfies a condition that the number of primary doctors to which the specialist have attended in the given period is smaller than the number of primary doctors to which the specialist can attend, at time when the matching request is obtained.

8. The medical service support method according to claim 2, wherein
the registration information includes information that identifies years of experience of each specialist, and
the identifying at least one specialist includes identifying as the at least one specialist, a specialist who has the years of experience equal to or longer than the years of experience identified in the matching request.

9. The medical service support method according to claim 2, wherein
the registration information includes information that identifies the number of primary doctors to which each specialist is attending, and
the identifying at least one specialist includes identifying the at least one specialist based on the number of primary doctors to which each specialist is attending at a time point when the matching request is obtained.

10. The medical service support method according to claim 2, wherein
the registration information includes information that identifies evaluation of each specialist, and
the identifying at least one specialist includes identifying the at least one specialist based on evaluation of each specialist.

11. The medical service support method according to claim 2, wherein
the registration information includes position information of each specialist, and
the identifying at least one specialist includes identifying the at least one specialist based on position information of the primary doctor from which the matching request has been obtained and the position information of each specialist.

12. A medical service support method comprising:
obtaining disease information on a disease from which a patient may be suffered, based on patient information provided by a primary doctor for the patient;
determining a reception of a matching request from the primary doctor;
allowing a plurality of specialists to view the disease information in response to the matching request from the primary doctor; and
receiving applications for an advisor from the plurality of specialists.

13. The medical service support method according to claim 12, wherein
the allowing a plurality of specialists to view the disease information includes allowing the plurality of specialists to view the patient information.

14. The medical service support method according to claim 1, further comprising providing the primary doctor with the disease information, wherein
the determining the reception of the matching request includes determining whether the matching request has been received through an information terminal from the primary doctor provided with the disease information.

15. A recording medium having a medical service support program stored thereon in a non-transitory manner, the medical service support program causing at least one processor to perform the medical service support method according to claim 1 by being executed by the at least one processor.

16. A medical service support system comprising:
at least one processor; and
a storage where a medical service support program is stored, wherein
the medical service support program causes the at least one processor to perform the medical service support method according to claim 1 by being executed by the at least one processor.
